(19) United States
(12) Patent Application Publication
(10) Pub. No.: US 2018/0251789 A1
(43) Pub. Date: Sep. 6, 2018

(54) MULTILAYER GENETIC SAFETY KILL CIRCUITS BASED ON SINGLE CAS9 PROTEIN AND MULTIPLE ENGINEERED GRNA IN MAMMALIAN CELLS

(71) Applicant: Massachusetts Institute of Technology, Cambridge, MA (US)

(72) Inventors: Samira Kiani, Somerville, MA (US); Ron Weiss, Newton, MA (US); Mohammad Reza Ebrahimkhani, Somerville, MA (US)

(73) Assignee: Massachusetts Institute of Technology, Cambridge, MA (US)

(21) Appl. No.: 15/755,623

(22) PCT Filed: Sep. 1, 2016
(86) PCT No.: PCT/US16/49907

§ 371 Date: Feb. 27, 2018

Related U.S. Application Data
(60) Provisional application No. 62/214,839, filed on Sep. 4, 2015.

(51) Int. Cl. C12N 15/90; C12N 15/63; C12N 9/22; A61K 48/00; C07H 21/02

(52) U.S. Cl. CPC C12N 15/907; C12N 15/63; C07H 21/02; A61K 48/00; C12N 9/22

(57) ABSTRACT

Aspects of the disclosure relate to synthetic regulatory systems composed of a multifunctional clustered regularly interspaced short palindromic repeat (CRISPR)-associated (Cas) nuclease and at least two distinct guide RNAs (gRNAs). The synthetic regulatory system modulates cleavage and transcription, including repression and activation, in a mammalian cell such as a human cell.

### RELATED APPLICATION

This application claims the benefit under 35 U.S.C. § 119(e) of U.S. provisional application No. 62/214,839, filed Sep. 4, 2015, which is incorporated by reference herein in its entirety.

### BACKGROUND OF INVENTION

Since its adaptation for site-specific DNA cleavage in 2012, the CRISPR-Cas9 system from Streptococcus pyogenes (SP-Cas9) has been widely used for genome editing in a variety of organisms, from prokaryotes to eukaryotes^{1,2}. By mutating residues involved in DNA catalysis, researchers have generated nuclease-null (dCas9) variants that retain the ability to bind DNA but lack endonucleolytic activity³. These dCas9 variants were later functionalized with effector domains such as transcriptional activation domains (ADs) or repression domains (RDs), enabling Cas9 to serve as a tool for programming transcriptional activity⁴⁻⁷.

### SUMMARY OF INVENTION

Cas9 is an RNA-guided DNA endonuclease that has been adopted for programmable genome editing and transcriptional regulation. Currently, no method exists to readily switch Cas9 between nuclease competent and nuclease null states. It has been demonstrated according to the invention that, by altering the length of the Cas9-associated guide RNA (gRNA), Cas9 nuclease activity can be controlled, enabling the simultaneous performance of genome editing and transcriptional activation or repression with a single Cas9 protein. These principles were exploited to engineer several mammalian synthetic circuits with combined transcriptional regulation and kill functions all governed by a single multifunctional Cas9 protein.

In some aspects, the invention is a synthetic regulatory system comprising a multifunctional Cas nuclease and at least two distinct gRNAs, wherein the synthetic regulatory system modulates cleavage and transcription in a mammalian cell such as a human cell. The system in some embodiments is a safety switch.

In some embodiments the two distinct gRNAs comprise a first gRNA of less than 15 nucleotides in length and a second gRNA of 15 or greater nucleotides in length. In other embodiments the first gRNA has a length of 10-14 nucleotides. In yet other embodiments the second gRNA has a length of 16-20 nucleotides.

A nucleic acid may encode the Cas nuclease and the at least two distinct gRNAs.

In some embodiments the Cas nuclease is fused to a transcriptional activation domain, such as VPR, or a transcriptional repression domain.

The synthetic regulatory system modulates cleavage and transcriptional activation in some embodiments. In other embodiments the synthetic regulatory system modulates cleavage and transcriptional repression. In yet other embodiments the synthetic regulatory system modulates cleavage, transcriptional repression and transcriptional activation.

A method for regulating a nucleic acid based therapeutic agent is also provided according to aspects of the invention. The method involves contacting a cell having a nucleic acid based therapeutic agent with a synthetic regulatory system comprising a multifunctional Cas nuclease and at least two distinct gRNAs, wherein the synthetic regulatory system modulates cleavage and transcription of the nucleic acid based therapeutic agent in the cell. The nucleic acid based therapeutic agent may be a DNA based vaccine, a gene therapy or a chimeric antigen receptor T cell (CAR-T) in some embodiments.

In other embodiments the synthetic regulatory system modulates cleavage and transcription of the nucleic acid based therapeutic agent in the cell when exposed to an exogenous factor. In yet other embodiments the synthetic regulatory system modulates cleavage and transcription of the nucleic acid based therapeutic agent in the cell when exposed to a cellular factor.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

These and other aspects of the invention, as well as various embodiments thereof, will become more apparent in reference to the drawings and detailed description of the invention.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1D show design and experimental analysis in human cells with multifunctional CRISPR devices and circuits. FIG. 1A is a schematic of a Cas9-VPR and 14nt gRNA repression device (top). EYFP output fluorescence was measured for samples transfected with or without 14nt gRNA vector and comparison was made with a dCas9/14nt gRNA-based repression device. Shown are geometric mean and s.d. of means of EYFP for cells expressing >10⁷ Molecules of Equivalent Fluorescein (MEFL) of transfection marker EBFP. n=3 independent technical replicates combined from three experiments (n=2 for +Cas9-VPR+gRNA). FIG. 1B is a schematic of parallel Cas9-VPR/14nt gRNA-based transcriptional repression and activation devices in a single cell. A 14nt gRNA-a drives Cas9-VPR to a CRISPR-activatable promoter (CAP) and mediates the activation of tdTomato while another 14nt gRNA targets Cas9-VPR to a CRISPR repressible promoter (CRP) to repress EYFP expression. Shown are geometric mean and s.d. of means of EYFP for cells expressing >10⁷ MEFL of transfection marker EBFP. n=4 independent technical replicates combined from three experiments. FIG. 1C shows schematics of a genetic kill switch designed to incorporate Cas9-VPR DNA cleavage and transcriptional activation functions. A 14nt gRNA directs Cas9-VPR to a CAP to activate output EYFP expression. Addition of doxycycline generates a 20nt gRNA that directs Cas9-VPR to the same region within the promoter, but cuts within the promoter, thereby decreasing EYFP output. In circuits containing dCas9-VPR instead, the same induction leads to enhanced activation. Shown are geometric mean and s.d. of means of EYFP for cells expressing >3×10⁷ MEFL of transfection marker EBFP. n=3 independent technical replicates combined from three experiments (n=2 for Cas9-VPR-dox). Left two bars: Cas9-VPR. Right two bars: dCas9-VPR. FIG. 1D illustrates a genetic kill circuit that incorporates all three functions of Cas9-VPR, DNA cleavage, transcriptional activation and repression. Input gRNA that cuts within TALER coding sequences decreases available gRNA-a and reduces output expression. Shown are geometric mean and s.d. of means of EYFP for cells expressing >10⁷ MEFL of transfection marker mKate. n=4 independent technical replicates combined from three experiments (n=2 in 48 h groups).

FIGS. 2A-2D are simplified schematics of the circuits in FIG. 1. FIG. 2A is a schematic of a Cas9-VPR and 14nt gRNA repression device. FIG. 2B is a schematic of parallel Cas9-VPR/14nt gRNA-based transcriptional repression and activation devices in a single cell. A 14nt gRNA-c drives Cas9-VPR to a CRISPR-activatable promoter (CAP) and mediates the activation of tdTomato while another 14nt gRNA targets Cas9-VPR to a CRISPR repressible promoter (CRP) to repress EYFP expression. FIG. 2C shows schematics of a genetic kill switch designed to incorporate Cas9-VPR DNA cleavage and transcriptional activation functions. A 14nt gRNA directs Cas9-VPR to a CAP to activate output EYFP expression. Addition of doxycycline generates a 20nt gRNA that directs Cas9-VPR to the same region within the promoter, but cuts within the promoter, thereby decreasing EYFP output. FIG. 2D illustrates a genetic kill circuit that incorporates all three functions of Cas9-VPR: DNA cleavage, transcriptional activation, and repression. Input gRNA cuts within TALER coding sequences, decreases available gRNA-a, and reduces output expression.

FIGS. 3A-3C show different promoter architectures used to analyze Cas9-VPR-medicated transcriptional repression. FIG. 3A shows schematics of Cas9-VPR/14nt gRNA based transcriptional repression control unit. FIG. 3B shows the architecture of different CRISPR Repressible Promoters (CRPs). FIG. 3C show the geometric mean and s.d. of means of EYFP for cells expressing >10⁷ MEFL of transfection marker EBFP (n=3 technical replicates). The highest repression was achieved using CRP-8. Some of the promoters designed for repression purposes unexpectedly led to activation, which require further analysis to understand the effect of spacing between Cas9-VPR target sites at the promoters or location of targeting (downstream of the promoter) on this observation.

FIGS. 4A-4E show the analysis of the dynamics of a genetic kill switch circuit. FIG. 4A is a schematic of a genetic kill switch designed such that 20nt and 14nt gRNAs compete for the same target site within a CAP (CRISPR-activatable promoter). Upon induction of 20nt gRNA and infrared fluorescent protein (iRFP) with doxycycline, reduction in EYFP expression is expected due to Cas9-VPR/20nt gRNA mediated cleavage within the CAP. FIG. 4B shows that 14ntgRNA/Cas9-VPR mediated activation of EYFP is detectable around 24 h post transfection and continues through 96 h. The control group received only the transfection marker EYFP and was measured 48 h post transfection. Shown are geometric mean and s.d. of means of EYFP for cells expressing >2×10⁷ MEFL of transfection marker EBFP. Bars, left to right: Control, 24 h, 48 h, 72 h, 96 h. FIG. 4C shows that, following addition of doxycycline, cells positive for iRFP and 20nt gRNA expression are detectable around 24 h after transfection and remain high in iRFP expression until 96 h. Shown are percent of cells expressing EBFP>10⁷ MEFL and iRFP>10^{6.5} relative to uninduced population. Each set of bars, left to right: 24 h, 48 h, 72 h, 96 h. FIG. 4D shows the fraction of cells that have EYFP above autofluorescence relative to uninduced population in different treatment conditions and overtime. Shown are percent of cells expressing EBFP>10⁷ MEFL and EYFP>10^{5.5} relative to uninduced population. Each set of bars, left to right: 24 h, 48 h, 72 h, 96 h. In FIG. 4E, the bars show the geometric mean ratio and standard deviation of mean ratio of uninduced vs. fully induced samples, for cells expressing >10⁷ MEFL of transfection marker EBFP. Group 1 includes cells that received doxycycline (4000 nM) at the time of transfection and group 2 includes cells that received doxycycline 24 h after the transfection. A slower dynamic was observed in group 2, possibly due to initial accumulation of EYFP protein. For all figures, n=3 independent technical replicates combined from three experiments. Each set of bars, left to right: 48 h, 96 h.

FIGS. 5A-5B provide insight into the design rules based on the concentrations of Cas9-VPR, 14nt gRNA, and 20nt gRNAs. FIG. 5A is a schematic of a genetic kill switch designed such that 20nt and 14nt gRNAs compete for the same target site within a CAP. FIG. 5B shows varying the dosages of transfected plasmids encoding Cas9-VPR, 14nt gRNA, 20nt-gRNA between low (5ng), medium (25ng for 14nt gRNA and 50ng for 20nt gRNA) and high (250ng) helps unravel some design rules. Each line represents a single condition of transfection with corresponding Cas9, 14nt gRNA, and 20nt gRNA plasmid levels in front of the fold change observed upon addition of doxycycline. Bar graphs show fold change of geometric mean and s.d. of means of EYFP over uninduced cells for cells expressing >3×10⁷ MEFL transfection marker EBFP. n=3 independent technical replicates combined from three experiments.

FIGS. 6A-6B show the design and analysis of a genetic kill switch that functions based on DNA cleavage in the Cas9-VPR coding sequence. FIG. 6A is a schematic of a genetic kill switch designed such that the presence of 20nt gRNAs leads to Cas9-VPR-mediated cleavage within its own coding sequence and thereby reverses the output EYFP and tdTomato levels. Comparing cells that either received 20nt gRNAs or did not, there is nearly a 5 fold de-repression of EYFP and about 1.4 fold decrease in tdTomato expression. Shown are geometric mean and s.d. of means of EYFP and tdTomato for cells expressing >10⁷ MEFL transfection marker EBFP. n=4 independent technical replicates combined from three experiments.

FIGS. 7A-7B show the design and analysis of a genetic kill switch that operates based on DNA cleavage in TALER coding sequence and reversal of a transcriptional repression device. FIG. 7A shows schematics of the kill switch involving a TALER-based transcriptional repression unit and Cas9-VPR mediated DNA mutation within TALER DNA sequence. FIG. 7B shows the geometric mean and s.d. of means of EYFP for cells expressing >10⁷ MEFL of transfection marker EBFP (n=3 technical replicates). T1-4 refer to 20nt gRNAs that cut at different regions in TALER coding sequence.

FIGS. 8A-8B show layering of the kill switch and cascade of the Cas9-VPR-mediated transcriptional repression devices. FIG. 8A is a schematic of the layered kill switch. FIG. 8B shows the geometric mean and s.d. of means of EYFP for cells expressing >10⁷ MEFL of transfection marker EBFP. Output is compared between cells with or without gRNA-encoding plasmids that cut within TALER coding sequences. (n=3 technical replicates).

### DETAILED DESCRIPTION

CRISPR technology has been widely applied for genome editing and modulation. The ease of engineering of the gRNA of the CRISPR system makes it an attractive platform for generating synthetic gene circuits and for synthetic biology purposes. A multifunctional CRISPR system has been applied here to engineer genetic circuits (simple and multi-layer) that can be used for generating safety off and kill switches with lesser genetic materials than available in the prior art. Thus, the technology of the invention involves the generation of circuits that can be packaged into delivery vehicles such as viruses that contain load limit for therapeutics, in a manner that could not be achieved using prior art methods.

With the development of gene and cell-based therapies that have revolutionized cancer therapy and other hard to treat diseases, there is an increasing need to develop and deliver additional regulatory mechanisms to control for specificity of these biological treatments or minimize off target effects. The synthetic biology-based genetic circuits of the invention provide significant advantages that allow for custom designs that are capable of incorporating multiple inputs/signals from environments and cells, leading to the generation of a desired outcome (intended by current gene or cell-based therapies, referred to herein as nucleic acid based therapeutic agents) after and only after receiving and processing those inputs/signals. Complex gene circuits have great utility because they allow better specificity and tighter controls for the desired therapeutic purposes such as safety switches. However, the more complex gene circuits become, the harder they become to engineer because of the metabolic load they create on cells. A single Cas9 protein with multiple functionality (cleaving and transcriptional activation/repression), such as that claimed herein, has great advantages because it provides better flexibility to engineer complex and multilayer genetic switches. These functionalities are achieved simply by altering and engineering gRNAs (which are small and easier to engineer).

Multi-layered and complex genetic off and kill switches in human cells using a Cas9 nuclease fused to a transcriptional activation domain (VPR), referred to as a multifunctional CRISPR was developed, as described in the examples below. A multi-layer and complex genetic kill switch, which is unique in human cells, can now be achieved using the technology of the invention. Truncating gRNA from the 5′ end decreases nuclease activity of Cas9-VPR complex while retaining its DNA binding capacity. Consequently, several genetic kill switches (and off switches) with increasing complexity were developed using shared and single Cas9-VPR and multiple gRNAs of different lengths. The complexity of the circuits achieved, the ease of engineering a lesser DNA footprint (a shared Cas9-VPR with multiple functions), and the use in therapeutic applications (safety switches) are highlights of the present invention.

A Cas nuclease is part of a CRISPR system. The components of the synthetic regulatory system of the invention may be in the form of one or more polynucleotide sequences. For instance, one or more polynucleotides may have sequences which encode a Cas nuclease, the at least 2 gRNAs (guide RNAs) and optionally a tracr sequence. When transcribed, the tracr mate sequence hybridizes to the tracr sequence and the guide sequence directs sequence-specific binding of a CRISPR complex to the target sequence. The polynucleotide sequence may be DNA or RNA or hybrids thereof.

A CRISPR enzyme is typically a type I or III CRISPR enzyme, preferably a type II CRISPR enzyme. The type II CRISPR enzyme may be any Cas enzyme. A preferred Cas enzyme is a Cas9 enzyme. The Cas9 enzyme may be from a spCas9 or saCas9 or may have a high degree of sequence homology with, a wildtype enzyme. The Cas enzyme can be any naturally-occurring Cas9 as well as any chimaeras, mutants, homologs or orthologs.

The polynucleotides may be under the control of a promoter, such as an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Still other types of inducible promoters which may be useful in this context are those which are regulated by a specific physiological state, e.g., temperature, acute phase, a particular differentiation state of the cell, or in replicating cells only.

The system of the invention may be used as a safety switch. For instance it may be used to control the expression and activity of intracellular nucleic acid target DNA in highly regulatable and precise manner. The target nucleic acid may be a nucleic acid or cellular therapy. The system of the invention can be designed to target DNA sequences of these therapeutics in order to reverse or shut down the effects of such therapeutics. In some instances the target DNA may be disrupted in its entirety in response to the methods of the invention. In some instances at least about 10%, 15%, 20%, 25%, 30%, 35%, 40%, 45%, or 50% of the target DNA is disrupted by use of the systems described herein. In some embodiments, at least about 60%, 70%, or 80% by of the target DNA is disrupted by administration of the systems of the invention. In some embodiments, at least about 85%, 90%, or 95% or more of the target DNA is disrupted.

The systems are useful for disrupting or interfering with the activity of therapeutics such as gene therapy, plasmid or viral vectors, cell based therapies such as CAR-Ts. Using the systems of the invention each of these therapies may be selectively turned off, for instance when the therapy is complete or if the subject has an adverse reaction to the therapy. Other uses are evident to the skilled artisan.

The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Example 1

### Cas9 gRNA Engineering for Selectable Genome Editing, Activation, and Repression

To date, no method exists that allows switching between Cas9 nuclease-dependent and -independent functions with relative ease. The ability for a single Cas9 protein to simultaneously perform genomic modifications while also modulating transcription would allow a user to gain control over two of the critical biomolecules in the cell, DNA and RNA. Such a tool would be transformative for a variety of applications, including therapeutic interventions, genetic screening, and synthetic genetic circuits¹⁻⁴.

In its native form, Cas9 is directed to a specific DNA sequence by a short guide RNA (gRNA) that contains 20 nucleotides (nt) complementary to its target. Truncated gRNAs, with 17 nt complementarity), have been shown to decrease undesired mutagenesis at some off-target sites without sacrificing on-target genome editing efficiencies⁵. In the same study, however, gRNAs containing <16nt showed a drastic reduction in nuclease activity. Analogous to earlier experiments examining the effects of increasing numbers of mismatches within the gRNA⁶, it was thought that the lack of DNA cleavage in the 16nt gRNA case is not due to a lack of DNA binding, but instead caused by inability of Cas9 to cleave the target substrate post-binding.

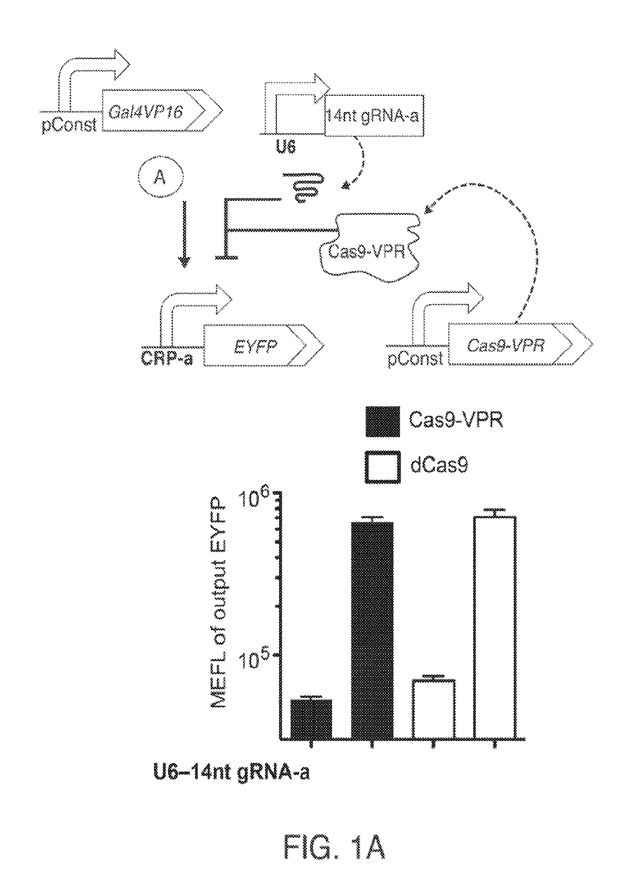

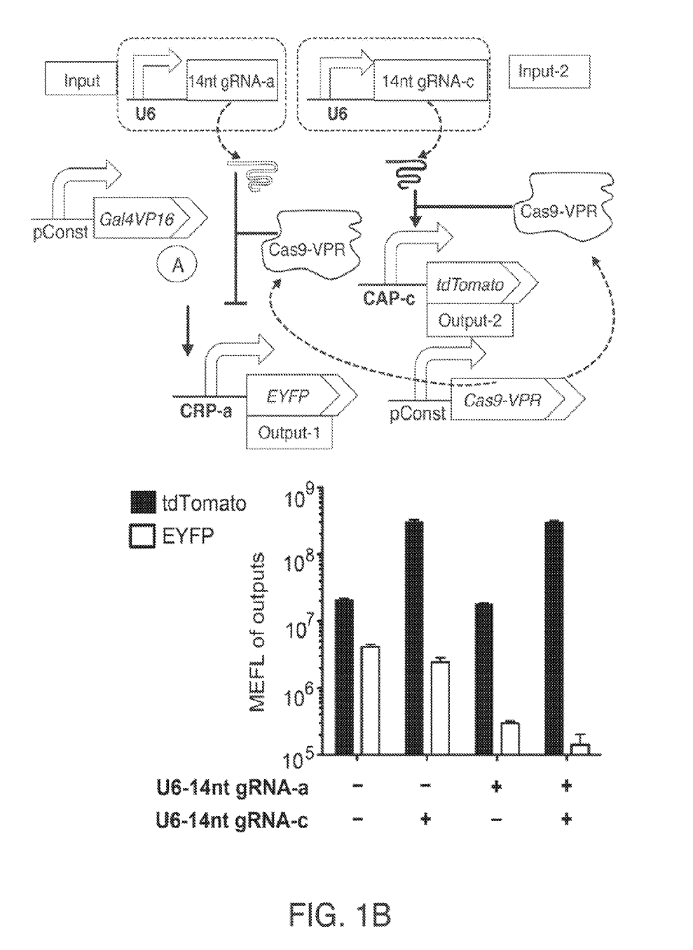

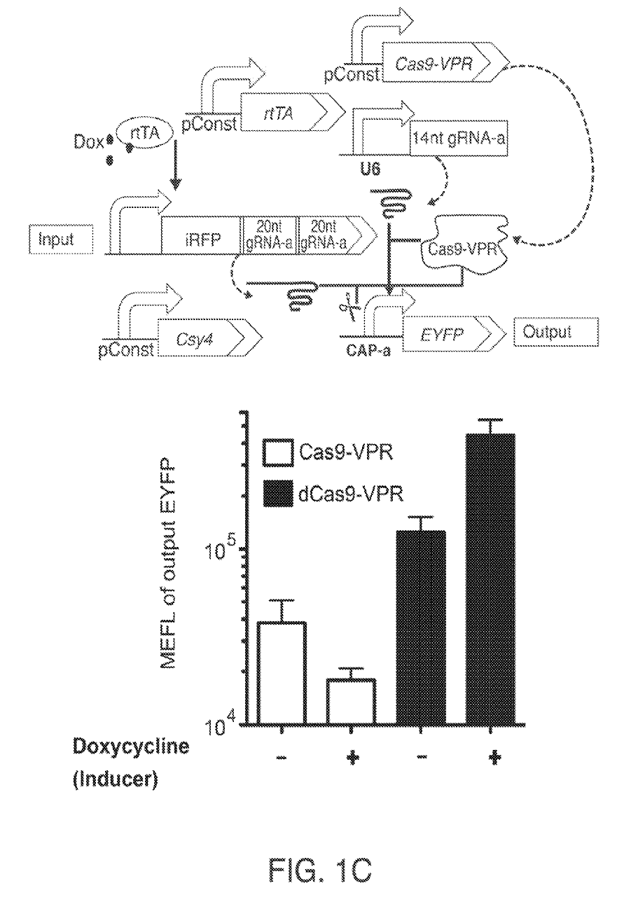

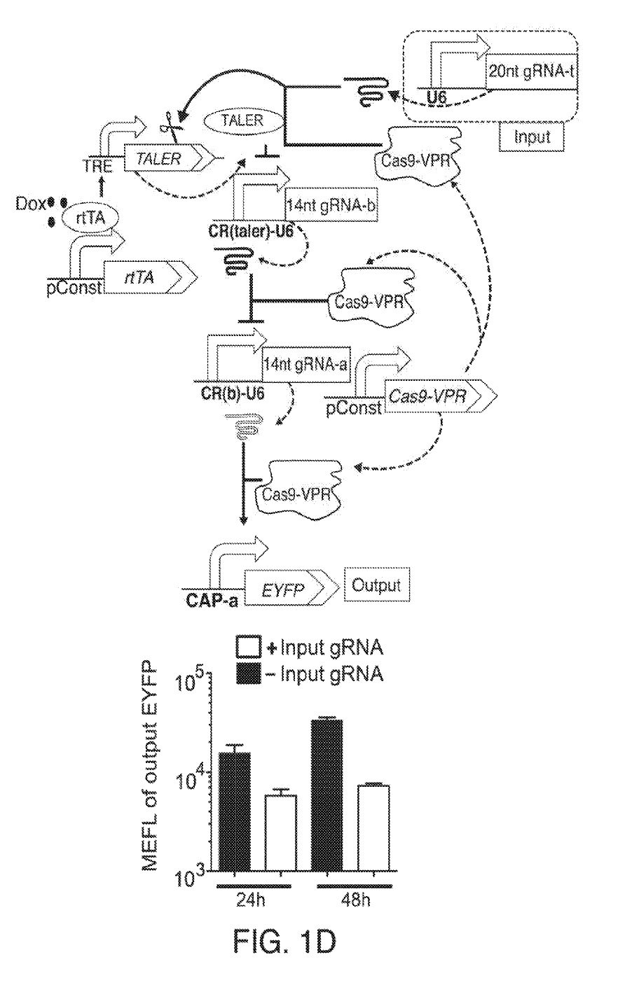

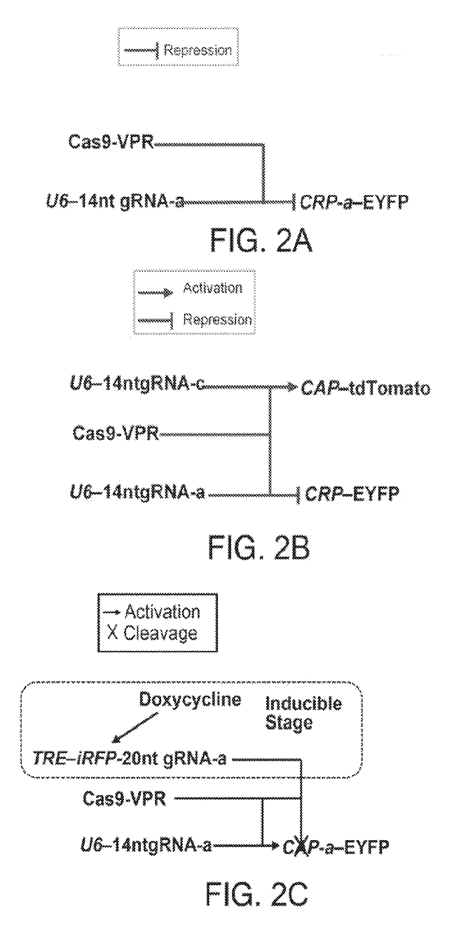

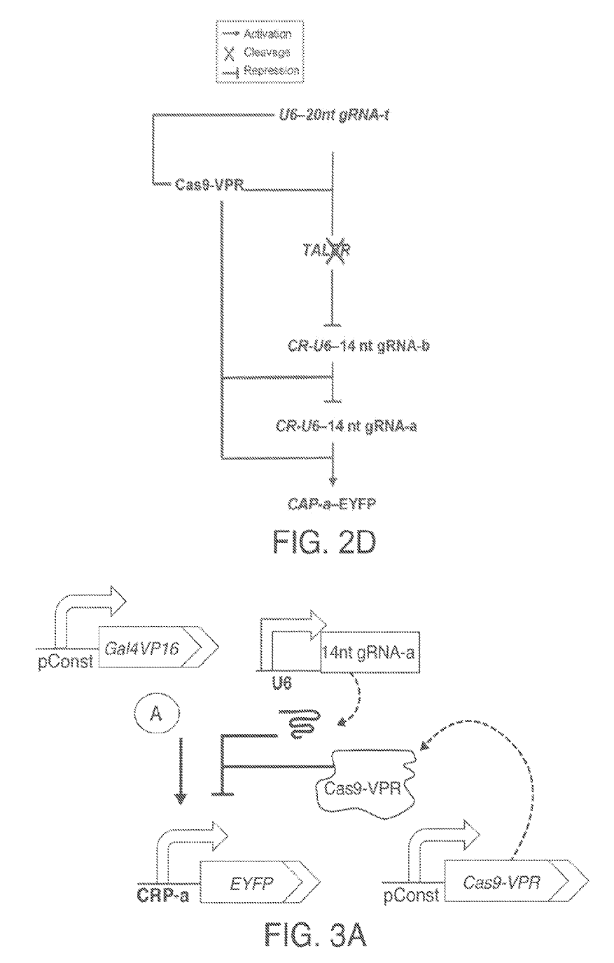

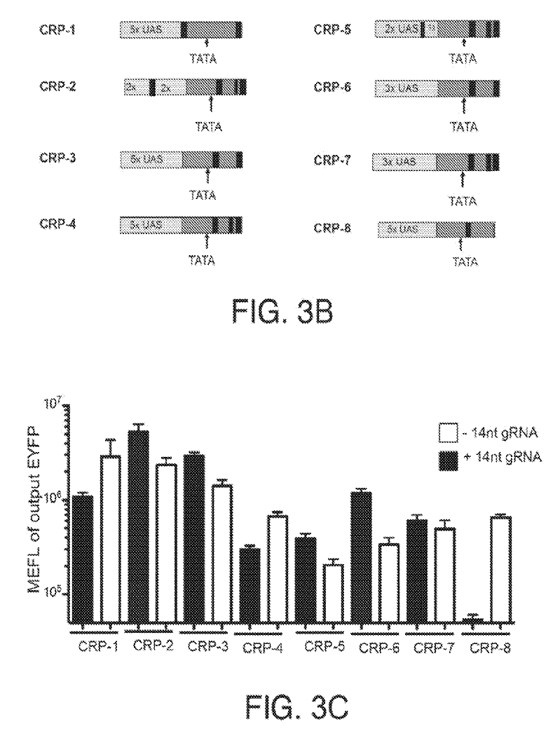

A number of synthetic transcriptional devices and layered circuits in human cells were generated using the multifunctional CRISPR system to test the feasibility of such system for synthetic biology purposes. A library of previously described CRISPR-repressible promoters (CRPs)⁹ was first developed in order to identify promoter architectures that allow efficient Cas9-VPR mediated transcriptional repression (FIGS. 2A, 3). A parallel experiment was then performed using the high-performance member of this promoter library (CRP-8, referred to as CRP-a in subsequent experiments) and similar repression efficiency (˜10-fold) was confirmed using dCas9 or Cas9-VPR with a 14nt gRNA to this promoter (FIG. 1A).

The use of Cas9-VPR and 14nt gRNAs in a single cell to achieve simultaneous transcriptional activation and repression was then evaluated. A CRP was placed upstream of Enhanced Yellow Protein (EYFP) and a CRISPR-activatable promoter (CAP) was placed upstream of tdTomato fluorescent protein, and were transfected into HEK293FT cells with other circuit regulatory elements. Flow cytometry analysis 48 h post transfection showed simultaneous repression and activation of fluorescent reporters (˜15-fold) were achieved with two 14nt gRNAs that target Cas9-VPR to the two promoters (FIGS. 1B, 2B).

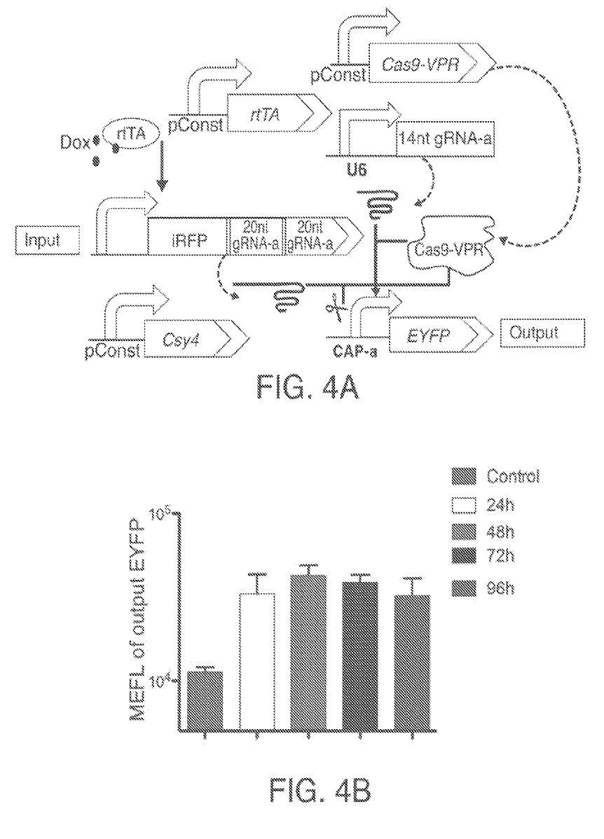

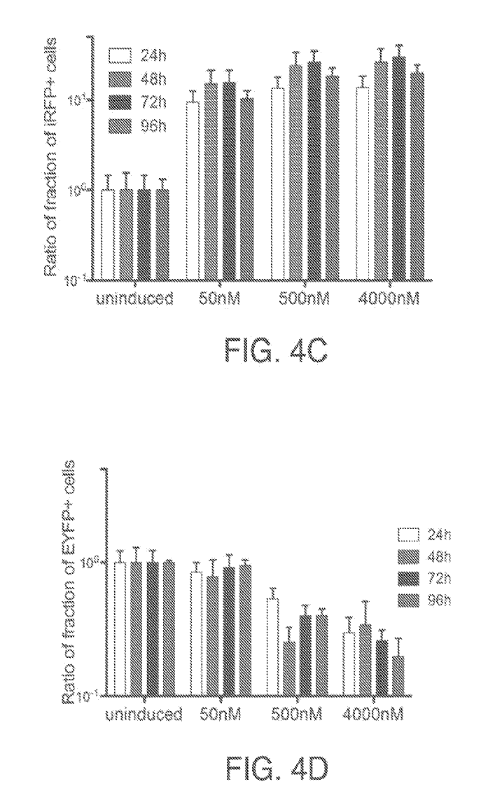

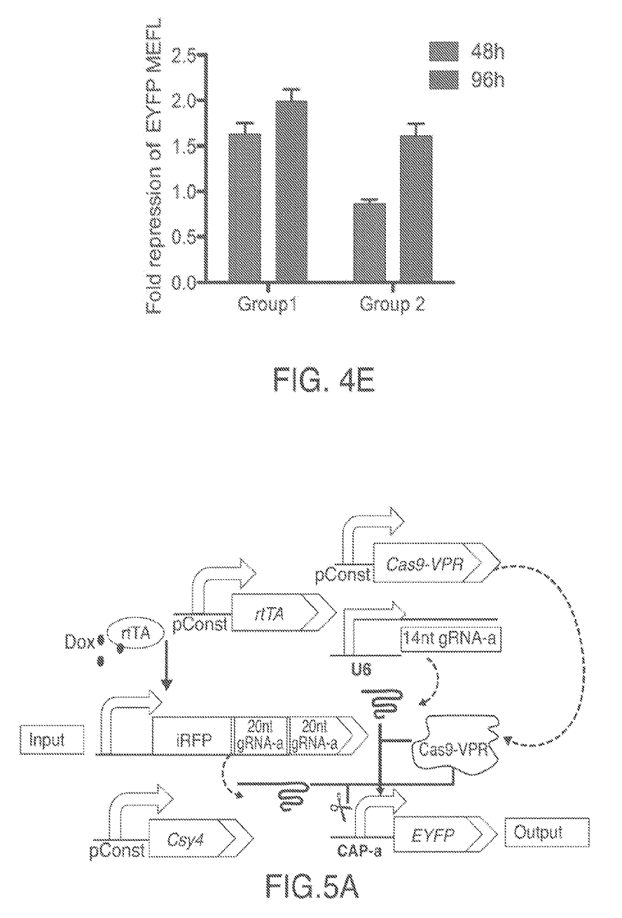

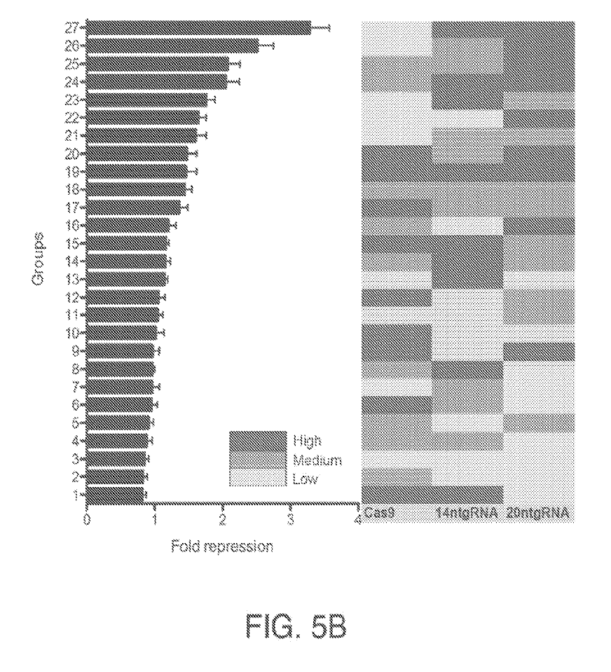

Subsequently, a genetic kill switch in which a 20nt gRNA that cuts within a CAP, is expressed under a tetracycline response element (TRE) promoter was designed^{9,10}. In the absence of a small molecule inducer (doxycycline), Cas9-VPR in combination with constitutively expressed 14nt gRNA for the same target within the CAP activates expression of EYFP. Upon addition of doxycycline, the 20nt guide enables Cas9-VPR to bind and cut within the CAP, leading to reduction of EYFP expression (FIGS. 1C, 2C). When a similar circuit was employed where Cas9-VPR was replaced with nuclease-null dCas9-VPR, doxycycline addition led to an increase rather than reduction of EYFP expression (FIG. 1C). Further analysis of this circuit revealed the dynamics and dosage response within this circuit topology (FIGS. 4-5).

A genetic kill switch design that operates by modulating the availability of Cas9-VPR within a cell was then tested. In the circuit, in the presence of a pair of full length 20nt gRNAs targeting the middle of the Cas9-VPR coding sequence, the guides directed Cas9-VPR to cut and disable itself and by doing so, decreased the available pool of Cas9-VPR within the cell, ultimately causing reduction of Cas9-VPR andl4nt gRNAs mediated inhibition or activation of the two fluorescent reporters (FIG. 14).

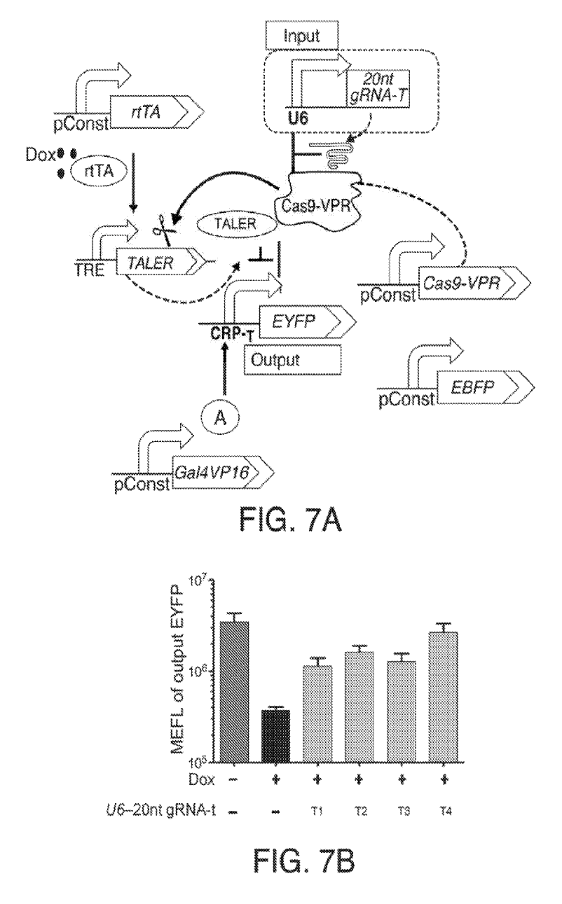

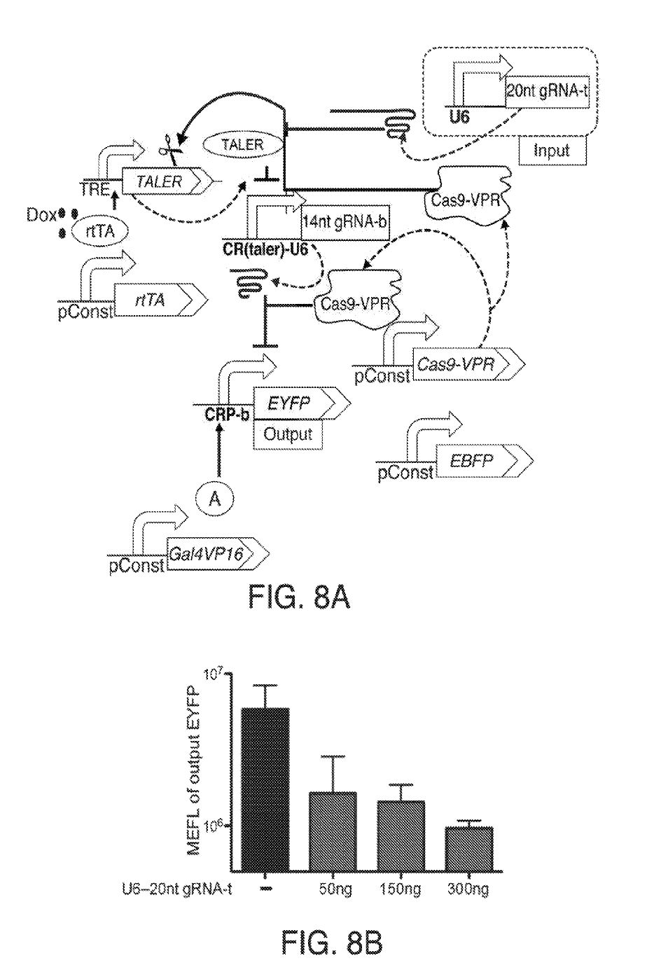

Next, progressively complex genetic kill switches that ultimately incorporate the three discussed functions of a single Cas9-VPR protein were designed and analyzed. To this end, one of the previously characterized Transcription Activator-Like Effector repressors (TALER)^{11,12} was employed, and whether Cas9-VPR could cleave within the TALER coding sequence and decrease available TALER, thus removing its repression of EYFP was examined (FIG. 7). A modified U6 promoter⁹ regulated by TALER was generated, which enabled one to connect the above genetic kill switch with a Cas9-VPR 14nt gRNA repression device. Transfection of this circuit in HEK293FT cells exhibited repression of output EYFP upon addition of input 20nt gRNAs that cut within the TALER coding sequence (FIG. 8). Finally, the genetic kill switch described in FIG. 8 was combined and interconnected with a Cas9-VPR-mediated transcriptional activation device to build a multilayered genetic circuit that simultaneously incorporates CRISPR-mediated transcriptional repression, activation, and DNA cleavage in a single circuit to modulate the output (FIGS. 1D, 2D). Flow cytometry analysis 24 and 48 h after transfection of HEK293FT revealed a functional circuit regulated by the input 20nt gRNA against TALER (FIG. 1D).

The ability of a single Cas9 protein to regulate RNA production while also maintaining the capacity to cleave DNA will be of great use in deciphering complex biological interactions and developing artificial genetic circuits. A promising use of the gRNA design principles will be in easily extending existing Cas9-based genome editing systems to concurrently modulate gene expression. This is particularly appealing in cases where considerable effort has been expended towards generating Cas9-expressing strains of mice or other labor-intensive and costly model systems^{13,14}. Further, the data suggests that nuclease-positive Cas9 can be easily endowed with other previously described dCas9 activities^{15,16} such as in vivo chromosomal tracking¹⁷ and facilitates the development of multifunctional synthetic genetic safety circuits with potential biomedical applications.

### Materials and Methods

### Fluorescent Reporter Assay for Quantifying Cas9 Activation

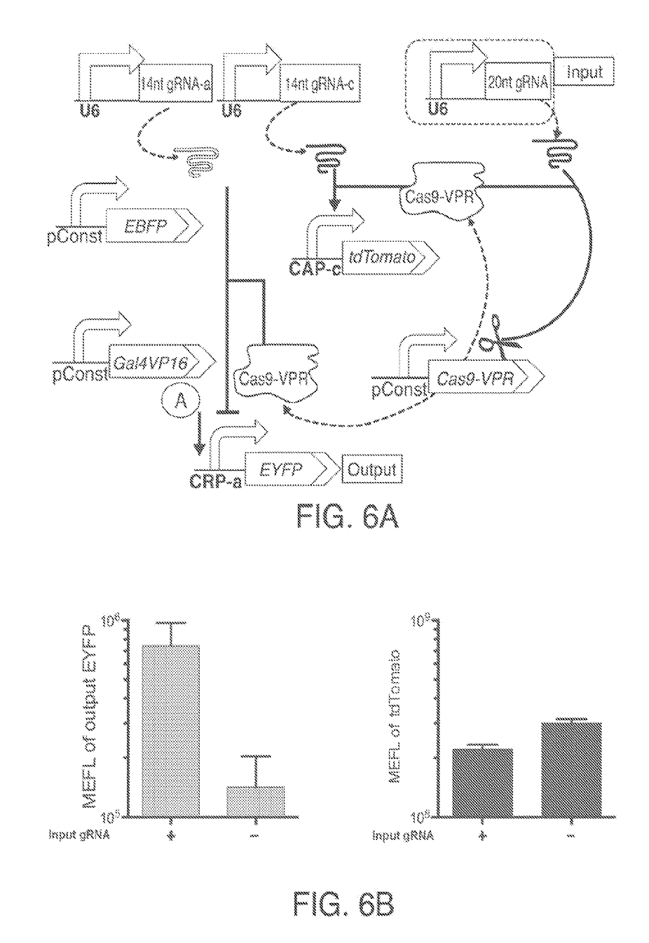

Fluorescent reporter experiments for FIGS. 3-4 were conducted with a plasmid (Addgene #47320) modified to include an extra gRNA binding site 100bp upstream of the already existing one. For ST1 and SA Cas9 experiments the protospacer remained the same but the PAM sequence was modified as needed for ST1 or SA Cas9. For FIG. 6, all experiments were conducted with a reporter with a single gRNA binding site. Reporter 1 denotes Addgene #47320, reporters 2 and 3 are similar to reporter 1 except the protospacer and PAM (in bold) were changed to contain the sequence GGGGCCACTAGGGACAGGATTGG (SEQ ID NO: 1) and AAGAGAGACAGTACATGCCCTGG (SEQ ID NO: 2) respectively. gRNAs of various length were co-transfected along with the indicated Cas9 protein and reporter into HEK293T cells along with an EBFP2 transfection control. Cells were analyzed by flow cytometry 48 hours post transfection and then when necessary were lysed to extract genomic DNA.

### Reporter Deletion Analysis

DNA was extracted using QuickExtract DNA Extraction Solution (Epicentre). DNA was then used for PCR to amplify desired regions. The amplified samples were then run on a 2% agarose gel stained with GelGreen (Biotium) and visualized using Gel Doc EZ (Bio-Rad). Band intensity was quantified using GelAnalyzer.

qRT-PCR Analysis

Samples were lysed and RNA was extracted using the RNeasy Plus Mini Kit (Qiagen). cDNA was made using the iScript cDNA synthesis kit (Bio-Rad) with 500ng of RNA. KAPA SYBR FAST Universal 2× qPCR Master Mix (Kapa Biosystems) was used for qPCR with 0.5 μl of cDNA used for each reaction. Activation was analyzed using CFX96 Real-Time PCR Detection System (Bio-Rad). Gene expression levels were normalized to β-actin levels.

### Endogenous Indel Analysis

DNA was extracted from 24-well plates using 350 μl of QuickExtract DNA Extraction Solution (Epicentre), according to the manufacturing instructions. Amplicon library preparation was performed using two PCRs. The first PCR to amplify from the genome, add appropriate barcodes and parts of adapters for Illumina sequencing. The second PCR extended out the Illumina adapters. In the first PCR, 5 μL of extracted DNA was used as template in a 100 μL Kapa HiFi PCR reaction and run for 30 cycles. PCR products were then purified using a homemade SPRI bead mixture and eluted in 50 μL of elution buffer. For the second PCR, 2 μL of the previous first round PCR was used as template in a 25 μL reaction and PCRs were run for a total of 9 cycles. PCR products were then run on an agarose gel, extracted and column purified. Equal amounts of each sample were then pooled and sequenced on an Illumina MiSeq using the paired end 150 MiSeq Nano kit.

Mate pair reads were merged into single contigs using FLASH¹⁸. Each contig was then mapped to a custom reference representing the three amplicons using bwa mem¹⁹. SAM output files were then converted to BAM files and pileup files were generated for each sample using SAM tools²⁰. Pileup files were then analyzed using custom python scripts to determine observed mutation rates. Mutations were only counted if the mutations spanned some portion of the sgRNA target site. In addition, base quality scores of ≥28 were also required for any mutations to be called. To minimize the impact of sequencing error, single base substitutions were excluded in this analysis.

### RNA Sequencing for Quantifying Activator Specificity

For each sample, 200 ng of total RNA was polyA selected using Dynabeads mRNA Purification Kit (Life Technologies). The RNA was then DNAse treated with Turbo DNase (Life Technologies) and cleaned up with Agencourt RNAClean XP Beads (Beckman Coulter). RNA-Seq Libraries were made using the NEBNext Ultra RNA Library Prep Kit for Illumina (New England BioLabs) according to the manufacturer's instructions with NEBNext Multiplex Oligos (New England BioLabs). Libraries were analyzed on a BioAnalyzer using a High Sensitivity DNA Analysis Kit (Agilent). Libraries were then quantified using a KAPA Library Quantification Kit (KAPA Biosystems) and pooled to a final concentration of 4 nM. Sequencing was performed on an Illumina NextSeq instrument with paired end reads. Reads were aligned to the hg19 UCSC Known Genes annotations using RSEM v1.2.1²¹ and analyzed in Python and R. Differential gene expression analysis was done using the Voom²² and Limma²³ packages in R for all genes with ≥1 TPM in each replicate, and a one-way within-subjects ANOVA was performed on the number of differentially expressed genes for each condition to quantify off-target effects, where differential expression was defined by Benjamini-Hochberg adjusted p-value<0.05 and fold-change>2 or <0.5. Raw RNA-seq data available at NCBI's Geo database: Accession number GSE70694.

### Statistical Analysis

All T-tests performed via GraphPad QuickCalcs Web Site graphpad.com/quickcalcs/ttest1/?Format=SEM (accessed June 2015.

### Cell Culture for Endogenous Target Mutation/Activation or Deletion Reporter

HEK-293T cells were cultivated in Dulbecco's Modified Eagle Medium (Life Technologies) with 10% FBS (Life Technologies) and Penicillin/Streptomycin (Life Technologies). Incubator conditions were 37° C. and 5% CO₂. Cells were tested for mycoplasma yearly. Cells were seeded into 24-well plates at 50,000 cells per well and transfected with 200ng of Cas9 construct, 10ng of guide, 60ng of reporter (for reporter experiments), 25ng of EBFP2 (for reporter experiments) via Lipofectamine 2000 (Life Technologies). Post transfection, cells were grown for 48-72 hours and lysed for either RNA or DNA extraction.

### Cell Culture for Circuit Experiments

Experiments were carried out in HEK293FT cells that were obtained from ATCC or were a gift from P. Mali, maintained in DMEM (CellGro) supplemented with 10% FBS (PAA Laboratories), 1% 1-glutamine-streptomycin-penicillin mix (CellGro) and 1% nonessential amino acids (NEAA; HyClone) at 37° C. and 5% CO₂ and tested for mycoplasma contamination. Transfections were performed using lipofectamine LTX and Attractene reagents (QIAGEN). Cells were seeded the day before transfection at 2×10⁵ cells per well in a 24-well plate. In control experiments, the DNA plasmid under study was replaced with an equivalent amount of empty DNA plasmid to maintain the total amount of transfected DNA constant among the groups. For transfections involving Attractene reagent, cocktails of plasmid DNAs were mixed and added to serum free DMEM to a total volume of 70 μl. 1.5-2 μl of Attractene was then added to each tube of DNA/DMEM mixtures, and the tube was gently mixed and kept at room temperature for 25 min to form the DNA-liposome complex. For experiments involving Lipofectamine LTX, cocktails of plasmid DNAs, serum-free DMEM and the Plus Reagent were mixed and incubated for 10 min. In parallel, LTX reagent was mixed with the serum-free media and incubated for the same period of time. After 10 min, the two reagents were mixed and incubated for an additional 30 min. Fresh medium was added to the cells directly before transfection (500 ml of DMEM with supplements). The DNA-reagent solution was then added drop-wise to the wells. Induction of the circuit was performed at this time as well by addition of doxycycline.

### Vector Design and Construction

Reporter gRNA was previously described (Addgene #48672), dCas9-VPR was previously described (Addgene #63798) and Cas9 was described (Addgene #41815). Cas9-VPR was cloned via Gateway assembly (Invitrogen) based on the Cas9 plasmid. gRNAs for endogenous targets were cloned into Addgene #41817 and transiently transfected. Plasmids used for synthetic circuits were constructed using the Gateway system. The U6-driven gRNA expression cassettes were ordered as blocks from IDT and cloned into a plasmid backbone using Golden Gate cloning. The library of CRPs were ordered as gene fragments from IDT and assembled into an appropriate promoter entry vector. Cas9-VPR Plasmids used in this study will be made available on Addgene (Addgene #68495,68496,68497,68498).

### Flow Cytometry for Circuit Experiments

Flow cytometry data were collected 48 h after transfection. Cells were trypsinized and centrifuged at 453 g for 5 min at 4° C. The supernatant was then removed, and the cells were resuspended in Hank's balanced salt solution without calcium or magnesium supplemented with 2.5% FBS. BD LSRII was used to obtain flow cytometry measurements with the following settings: EBFP, measured with a 405 nm laser and a 450/50 filter; EYFP, measured with a 488 nm laser and a 530/30 filter; tdTomato, measured with a 561 nm laser and a 695/40 filter. Non-transfected controls were included in each experiment. Data shown in the figures are geometric mean and s.d. of means for cells expressing the transfection marker EBFP. Sample sizes were predetermined for each experiment based on initial pilot experiments. At least 100,000 flow cytometry events were gathered per biological replicate.

### Statistical Analysis for Circuit Experiments

Flow cytometry data were converted from arbitrary units to compensated molecules of equivalent fluorescein (MEFL)²⁴ using the Tool-Chain to Accelerate Synthetic Biological Engineering (TASBE) characterization²⁵(MIT CSAIL Tech. Report 2012-008 (2012). An affine compensation matrix is computed from single positive and blank controls. FITC measurements are calibrated to MEFL using SpheroTech RCP-30-5-A beads, and mappings from other channels to equivalent FITC are computed from co-transfection of constitutive blue, yellow and red fluorescent proteins, each controlled by the CAG promoter on its own otherwise identical plasmid. Non-transfected controls were included in each experiment. Sample sizes were pre-determined for each experiment. Data shown in the figures are geometric mean and s.d. of means for cells expressing the transfection marker enhanced blue fluorescent protein (EBFP) based on the MEFL threshold set. More precisely, a threshold as a cutoff for each data set was selected based on the observed constitutive fluorescence distributions, and data below that threshold was excluded as being too close to the non-transfected population. Then the MEFL data was divided by constitutive fluorescent protein expression into logarithmic bins at 10 bins per decade, and the geometric mean and variance for those data points in each bin were calculated. High outliers were removed by excluding bins without at least 100 data points. In fact, both population and per-bin geometric statistics were calculated using this filtered set of data. Exclusion criteria for samples during flow cytometry analysis are the following predetermined criteria: samples containing less than 10% of the number of events or less than 10% of the fraction of successful transfections of the mode for the batch in which they were collected.

### Reproducibility

Sample sizes for each experiment were chosen based on an initial pilot experiment and were further guided by sample sizes from similar experiments and publications. No randomization or blinding was used in the course of the experiments. No data were excluded from analysis.

### REFERENCES

1. Hsu, P. D., E. S. Lander, and F. Zhang, Cell 157 (6), 1262-1278 (2014).
2. Sander, J. D. and J. K. Joung, Nature biotechnology 32 (4), 347-355 (2014).
3. Qi, L. S. et al., Cell 152 (5), 1173-1183 (2013).
4. Mali, P., K. M. Esvelt, and G. M. Church, Nature methods 10 (10), 957-963 (2013).
5. Fu, Y., J. D. Sander, D. Reyon, V. M. Cascio, and J. K. Joung, Nature biotechnology 32 (3), 279-284 (2014).
6. Jiang, W., D. Bikard, D. Cox, F. Zhang, and L. A. Marraffini, Nature biotechnology 31 (3), 233-239 (2013).
7. Chavez, A. et al., Nature methods 12 (4), 326-328 (2015).
8. Esvelt, K. M. et al., Nature methods 10 (11), 1116-1121 (2013).
9. Kiani, S. et al., Nature methods 11 (7), 723-726 (2014).
10. Nissim, L., S. D. Perli, A. Fridkin, P. Perez-Pinera, and T. K. Lu, Molecular cell 54 (4), 698-710 (2014).
11. Li, Y. et al., Nature chemical biology 11 (3), 207-213 (2015).
12. Davidsohn, N. et al., ACS synthetic biology 4 (6), 673-681 (2015).
13. Dow, L. E. et al., Nature biotechnology 33 (4), 390-394 (2015).
14. Platt, R. J. et al., Cell 159 (2), 440-455 (2014).
15. Perez-Pinera, P. et al., Nature methods 10 (10), 973-976 (2013).
16. Maeder, M. L. et al., Nature methods 10 (10), 977-979 (2013).
17. Chen, B. et al., Cell 155 (7), 1479-1491 (2013).
18. Magoc, T. and S. L. Salzberg, Bioinformatics 27 (21), 2957-2963 (2011).
19. Li, H. and R. Durbin, Bioinformatics 25 (14), 1754-1760 (2009).
20. Li, H. et al., Bioinformatics 25 (16), 2078-2079 (2009).
21. Li, B. and C. N. Dewey, BMC bioinformatics 12, 323 (2011).
22. Law, C. W., Y. Chen, W. Shi, and G. K. Smyth, Genome biology 15 (2), R29 (2014).
23. Smyth, G. K., Statistical applications in genetics and molecular biology 3, Article 3 (2004).
24. Hoffman, R. A., L. Wang, M. Bigos, and J. P. Nolan, Cytometry. Part A: the journal of the International Society for Analytical Cytology 81 (9), 785-796 (2012).
25. Beal, J., Frontiers in bioengineering and biotechnology 2, 87 (2014).

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A synthetic regulatory system comprising a multifunctional Cas nuclease and at least two distinct gRNAs, wherein the synthetic regulatory system modulates cleavage and transcription in a mammalian cell.

2. The synthetic regulatory system of claim 1, wherein the system is a safety switch.

3. The synthetic regulatory system of claim 1 or 2, wherein the two distinct gRNAs comprise a first gRNA of less than 15 nucleotides in length and a second gRNA of 15 or greater nucleotides in length.

4. The synthetic regulatory system of claim 3 wherein the first gRNA has a length of 10-14 nucleotides.

5. The synthetic regulatory system of claim 3, wherein the second gRNA has a length of 16-20 nucleotides.

6. The synthetic regulatory system of any one of claims 1-5, wherein a nucleic acid encodes the Cas nuclease and the at least two distinct gRNAs.

7. The synthetic regulatory system of any one of claims 1-5, wherein the Cas nuclease is fused to a transcriptional activation domain.

8. The synthetic regulatory system of claim 7, wherein the transcriptional activation domain is VPR.

9. The synthetic regulatory system of any one of claims 1-5, wherein the synthetic regulatory system modulates cleavage and transcriptional activation.

10. The synthetic regulatory system of any one of claims 1-5, wherein the synthetic regulatory system modulates cleavage and transcriptional repression.

11. The synthetic regulatory system of any one of claims 1-5, wherein the synthetic regulatory system modulates cleavage, transcriptional repression and transcriptional activation.

12. A method for regulating a nucleic acid based therapeutic agent, comprising contacting a cell having a nucleic acid based therapeutic agent with a synthetic regulatory system comprising a multifunctional Cas nuclease and at least two distinct gRNAs, wherein the synthetic regulatory system modulates cleavage and transcription of the nucleic acid based therapeutic agent in the cell.

13. The method of claim 12, wherein the nucleic acid based therapeutic agent is a DNA based vaccine.

14. The method of claim 12, wherein the nucleic acid based therapeutic agent is a gene therapy.

15. The method of claim 12, wherein the nucleic acid based therapeutic agent is a chimeric antigen receptor T cell (CAR-T).

16. The method of any one of claims 12-15, wherein the synthetic regulatory system modulates cleavage and transcription of the nucleic acid based therapeutic agent in the cell when exposed to an exogenous factor.

17. The method of any one of claims 12-15, wherein the synthetic regulatory system modulates cleavage and transcription of the nucleic acid based therapeutic agent in the cell when exposed to a cellular factor.


SEQUENCE LISTING (2 sequences)

SEQ ID NO: 1 - DNA, 23 bp, Artificial Sequence
  protospacer and PAM
ggggccactagggacaggattgg

SEQ ID NO: 2 - DNA, 23 bp, Artificial Sequence
  protospacer and PAM
aagagagacagtacatgccctgg